(19) United States
(12) Patent Application Publication
(10) Pub. No.: US 2003/0129713 A1
(43) Pub. Date: Jul. 10, 2003

(54) Process for preparing enantiomer-enriched cyanohydrins using acetals or ketals as substrates

(76) Inventors: Wolfgang Skranc, Wien (AT); Peter Poechlauer, Linz (AT); Irma Wirth, Enns (AT); Rudolf Neuhofer, Mittertreffling (AT); Herbert Mayrhofer, Engerwitzdorf (AT)

Correspondence Address: WENDEROTH, LIND & PONACK, L.L.P., 2033 K STREET N. W., SUITE 800, WASHINGTON, DC, 20006-1021, US

(21) Appl. No.: 10/326,065

(22) Filed: Dec. 23, 2002

(30) Foreign Application Priority Data
Dec. 27, 2001 (AT) A 2032/2001

(51) Int. Cl. C12P 17/00; C12P 13/00; C12N 9/88

(52) U.S. Cl. 435/117; 435/128; 435/232

(57) ABSTRACT

Process for preparing enantiomer-enriched cyanohydrins in which an acetal or ketal of the formula (I) is reacted

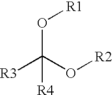

where

R1 and R2 independently of one another are an unsubstituted or substituted C₁-C₂₀-alkyl, C₅-C₂₀-aryl or C₅-C₂₀-heteroaryl radical,

or one of the two radicals is hydrogen, or R1 and R2 together form an unsubstituted or substituted C₂-C₂₀-alkylene radical,

and R3 and R4 independently of one another can be an unsubstituted or substituted C₁-C₂₀-alkyl, C₅-C₂₀-aryl, C₅-C₂₀-heteroaryl, C₇-C₂₀-alkylaryl, C₅-C₂₀-alkyl heteroaryl or C₅-C₂₀-aralkyl radical or an unsubstituted or substituted C₅-C₂₀-heterocycle or C₅-C₂₀-alkyl heterocycle or together can be an unsubstituted or substituted C₄-C₂₀-alkylene radical which can contain one or more heteroatoms in the chain, or one of the radicals is hydrogen,

in the presence of an (R)- or (S)-hydroxynitrile lyase and a cyanide group donor in an organic, aqueous or two-phase system or in emulsion at −5 to +40° C. to give the corresponding enantiomer-enriched cyanohydrins of the formula (II),

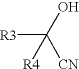

where

R3 and R4 are as defined above.

Cyanohydrins are of importance for the synthesis of alpha-hydroxy acids, alpha-hydroxy ketones, beta-amino alcohols which are used to produce biologically active substances, for example active pharmaceutical compounds, vitamins, or else pyrethroid compounds.

These cyanohydrins are prepared by adding prussic acid to the carbonyl group of a ketone or aldehyde.

From the literature, a plurality of process variants are already known which describe the preparation of (R)- and/or (S)-cyanohydrins from aliphatic, aromatic or heteroaromatic aldehydes or else from aliphatic or aromatic ketones.

Thus EP-A-0 326 063 claims an enzymatic process for preparing optically active (R)- or (S)-cyanohydrins by reacting aliphatic, aromatic or heteroaromatic aldehydes or ketones with prussic acid in the presence of (R)-oxynitrilase (EC 4.1.2.10) from Prunus amygdalus or oxynitrilase (EC 4.1.2.11) from Sorghum bicolor. Acetals and ketals are not described, however. EP 0 632 130 further describes a process in which aliphatic aldehydes or unsymmetric aliphatic ketones are reacted with prussic acid and oxynitrilase from Hevea brasiliensis in a stereospecific manner to give (S)-cyanohydrins. Acetals and ketals are not mentioned therein.

EP 0 927 766 describes an enzymatic process for preparing optically active (S)-cyanohydrins from aliphatic, aromatic or heteroaromatic aldehydes or ketones in an emulsion. Acetals and ketals as substrates are not described therein.

EP 0 528 256 indicates that hydroxypivaldehyde can only be reacted using greatly elevated enzyme concentration and under suitable conditions to give D-2,4-dihydroxy-3,3-dimethylbutanonitrile with enantiomeric excesses of up to 81.4% ee. In the case of other previously known processes which do not use greatly elevated amounts of enzyme, as described in EP 0 528 256, an enantiomeric excess is not obtained, or an insufficiently great enantiomeric excess is obtained. One reason for this is, inter alia, also the fact that the substrate hydroxypivaldehyde is unstable.

Since many carbonyl compounds, for instance hydroxyaldehydes, which cyclize or polymerize spontaneously, are unstable, it was an object of the invention to find a process which is suitable, in particular, for preparing optically active cyanohydrins, the corresponding carbonyl compounds of which are unstable under previously known conditions, the cyanohydrins being obtained in good yields and with greater enantiomeric excess compared with reactions involving free aldehydes or ketones as substrate.

Unexpectedly, this object was achieved by using acetals or ketals as substrates.

The invention therefore relates to a process for preparing enantiomer-enriched cyanohydrins using hydroxynitrile lyase (HNL) and a cyanide group donor which comprises reacting an acetal or ketal of the formula (I),

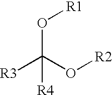

where

R1 and R2 independently of one another are an unsubstituted, monosubstituted or polysubstituted C₁-C₂₀-alkyl, C₅-C₂₀-aryl or C₅-C₂₀-heteroaryl radical,

or one of the two radicals is hydrogen, or R1 and R2 together form an unsubstituted, monosubstituted or polysubstituted C₂-C₂₀-alkylene radical,

and R3 and R4 independently of one another can be an unsubstituted, monosubstituted or polysubstituted C₁-C₂₀-alkyl, C₅-C₂₀-aryl, C₅-C₂₀-heteroaryl, C₇-C₂₀-alkylaryl, C₅-C₂₀-alkyl heteroaryl or C₅-C₂₀-aralkyl radical or an unsubstituted, monosubstituted or polysubstituted C₅-C₂₀-heterocycle or C₅-C₂₀-alkyl heterocycle or together can be an unsubstituted or substituted C₄-C₂₀-alkylene radical which can contain one or more heteroatoms in the chain, or one of the radicals is hydrogen,

in the presence of an (R)- or (S)-hydroxynitrile lyase and a cyanide group donor in an organic, aqueous or two-phase system or in emulsion at a temperature of −5 to +40° C. to give the corresponding enantiomer-enriched cyanohydrins of the formula (II),

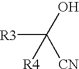

where

R3 and R4 are as defined above.

In the inventive process, acetals or ketals of the formula (I) are used as substrates.

In the formula (I) R1 and R2 independently of one another can be an unsubstituted, monosubstituted or polysubstituted C₁-C₂₀-alkyl, C₅-C₂₀-aryl or C₅-C₂₀-heteroaryl radical.

A C₁-C₂₀-alkyl radical is taken to mean here saturated or monounsaturated or polyunsaturated, linear, branched or cyclic, bridged, primary, secondary or tertiary hydrocarbon radicals. These are, for example, methyl, ethyl, propyl, isopropyl, propenyl, butyl, isobutyl, t-butyl, butenyl, butinyl, pentyl, cyclopentyl, isopentyl, neopentyl, pentenyl, hexyl, isohexyl, cyclohexyl, cyclohexylmethyl, 3-methylpentyl, 2,2-dimethylbutyl, 2,3-dimethylbutyl, octyl, cyclooctyl, decyl, cyclodecyl, dodecyl, cyclododecyl etc.

Preference is given here to C₁-C₁₂-alkyl radicals, and particular preference to C₁-C₄-alkyl radicals.

Aryl is taken to mean preferably C₆-C₂₀-aryl groups, for instance phenyl, biphenyl, naphthyl, indenyl, fluorenyl etc.

Heteroaryl is preferably taken to mean cyclic radicals having from 6 to 20 carbon atoms which contain at least one S, O or N atom in the ring.

The radicals can be unsubstituted or substituted by one or more substituents selected from the group consisting of phenyl, C₁-C₆-alkyl, OH, halogen or sulfoxy.

In this case full acetals are used as substrates.

Preferably, R1 and R2 are an alkyl radical.

If cyclic acetals are used as substrates, R1 and R2 together form a C₂-C₂₀-alkylene radical which can be unsubstituted or substituted by one or more substituents from the group consisting of phenyl, C₁-C₆-alkyl, OH, halogen or sulfoxy.

Suitable substrates are also semiacetals in which one of the radicals R1 or R2 is hydrogen.

Particularly preferably, R1 and R2 independently of one another are a saturated, linear or branched C₁-C₄-alkyl radical, or one of the two radicals is hydrogen, or R1 and R2 together form a C₂-C₆-alkylene radical which can be unsubstituted or substituted by one or more substituents selected from the group consisting of C₁-C₄-alkyl or OH.

R3 and R4, in the formula (I), are independently of one another an unsubstituted, monosubstituted or polysubstituted C₁-C₂₀-alkyl, C₅-C₂₀-aryl, C₅-C₂₀-heteroaryl, C₇-C₂₀-alkylaryl, C₅-C₂₀-alkyl heteroaryl or C₅-C₂₀-aralkyl radical or an unsubstituted, monosubstituted or polysubstituted C₅-C₂₀-heterocycle or C₅-C₂₀-alkyl heterocycle.

C₁-C₂₀-alkyl again is taken to mean saturated or monounsaturated or polyunsaturated, linear, branched or cyclic, bridged, primary, secondary or tertiary hydrocarbon radicals, for instance methyl, ethyl, propyl, isopropyl, propenyl, butyl, isobutyl, t-butyl, butenyl, butinyl, pentyl, cyclopentyl, isopentyl, neopentyl, pentenyl, hexyl, isohexyl, cyclohexyl, cyclohexylmethyl, 3-methylpentyl, 2,2-dimethylbutyl, 2,3-dimethylbutyl, octyl, cyclooctyl, decyl, cyclodecyl, dodecyl, cyclododecyl etc.

Preference is given here to C₁-C₁₂-alkyl radicals, and particular preference to C₁-C₈-alkyl radicals. The alkyl group can be unsubstituted, monosubstituted or polysubstituted by groups inert under the reaction conditions. Suitable substituents are, for example, unsubstituted or substituted aryl or heteroaryl groups, such as phenyl, phenoxy or indolyl groups, halogen, hydroxyl, hydroxy-C₁-C₅-alkyl, C₁-C₆-alkoxy, aryloxy, preferably C₆-C₂₀-aryloxy, C₁-C₆-alkylthio, amino, alkylamino, preferably C₁-C₆-alkylamino, arylamino, preferably C₆-C₂₀-arylamino, ether, thioether, carboxylic ester, carboxamide, sulfoxide, sulfone, sulfonic acid, sulfonic ester, sulfinic acid, mercaptan, nitro or azido groups.

Aryl is preferably taken to mean C₆-C₂₀-aryl groups, for instance phenyl, biphenyl, naphthyl, indenyl, fluorenyl, etc.

The aryl group can here be unsubstituted, monosubstituted or polysubstituted. Suitable substituents are again unsubstituted or substituted aryl or heteroaryl groups, such as phenyl, phenoxy or indolyl groups, halogen, hydroxyl, hydroxy-C₁-C₅-alkyl, C₁-C₆-alkoxy, aryloxy, preferably C₆-C₂₀-aryloxy, C₁-C₆-alkylthio, amino, alkylamino, preferably C₁-C₆-alkylamino, arylamino, preferably C₆-C₂₀-arylamino, ether, thioether, carboxylic ester, carboxamide, sulfoxide, sulfone, sulfonic acid, sulfonic ester, sulfinic acid, mercaptan, nitro or azido groups.

Alkaryl or alkylaryl are taken to mean alkyl groups which have an aryl substituent.

Aralkyl or arylalkyl relates to an aryl group having an alkyl substituent.

Heteroaryl or heterocycle is taken to mean cyclic radicals which contain at least one S, O or N atom in the ring. These are, for example, furyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzoimidazolyl, purinyl, carbazolyl, oxazolyl, thiazolyl, isothiazolyl, 1,2,4-thiadiazolyl, isoxazolyl, pyrrolyl, quinazolinyl, pyradazinyl, phthalazinyl, morpholinyl, etc.

Functional O or N groups can be protected here if required.

The heteroaryl group or the heterocycle can be unsubstituted, monosubstituted or polysubstituted by the substituents already set forth above.

Alkyl heteroaryl or alkyl heterocycle is taken to mean here alkyl groups which are substituted by a heteroaryl group or by a heterocycle.

Preferably, R3 and R4 are a saturated or unsaturated, linear or branched C₁-C₈-alkyl radical, a benzyl or a phenyl radical, where the radicals can be unsubstituted, monosubstituted or polysubstituted by OH, halogen, phenyl, carboxylic acid derivatives, such as carboxylic esters or carboxamides, amino, C₁-C₆-alkylamino, C₆-C₂₀-arylamino, C₁-C₆-alkoxy, C₆-C₂₀-aryloxy, or nitro.

R3 and R4, however, together can also be an unsubstituted or substituted C₄-C₂₀-alkylene radical which can contain in the chain one or more heteroatoms selected from the group consisting of O, N or S, or an NR5R6 group, where R5 and R6 independently of one another can be H or C₁-C₆-alkyl.

Preference is given to C₄-C₇-alkylene radicals which, depending on the ring size of the cyclic ketone, have at most two heteroatoms in the alkyl chain. The alkylene chain can, in addition, again depending on the ring size, have one or two double bonds. The alkylene radical can in addition be monosubstituted or polysubstituted by the radicals set forth above.

However, in the starting materials used, one of the radicals R3 and R4 can also be hydrogen.

In the inventive process, acetals or ketals of the formula (I), some of which are commercially available, or can be prepared, for example, in a similar manner to Synthesis 1981, 501-522, are reacted to form enantiomer-enriched (R)- and (S)-cyanohydrins.

The corresponding acetals or ketals of the formula (I) are reacted in the presence of a cyanide group donor with an (R)- or (S)-hydroxynitrile lyase, the acetals or ketals being cleaved in situ to give the corresponding aldehyde or ketone and alcohol, and the aldehyde or the ketone reacting under enzyme catalysis to give the corresponding enantiomer-enriched cyanohydrin.

Suitable cyanide group donors are prussic acid, alkali metal cyanides or cyanohydrins of the general formula (III)

R7R8C(OH)(CN).

In the formula (III) R7 and R8 independently of one another are hydrogen or an unsubstituted hydrocarbon group, or R7 and R8 together are an alkylene group having 4 or 5 carbon atoms, where R7 and R8 are not simultaneously hydrogen. The hydrocarbon groups are aliphatic or aromatic groups, preferably aliphatic groups. Preferably, R7 and R8 are alkyl groups having 1-6 carbon atoms, and particularly preferably acetone cyanohydrin is the cyanide group donor of the formula (III).

The cyanide group donor can be prepared by known processes. Cyanohydrins, in particular acetone cyanohydrin, are also commercially available.

Preferably, the cyanide group donor is prussic acid, KCN, NaCN or acetocyanohydrin, particularly preferably prussic acid.

The prussic acid can also be released here just shortly before the reaction from one of its salts, for instance NaCN or KCN, and added to the reaction mixture without solvent or in dissolved form.

Per mol of acetal or ketal used, at least 1 mol, preferably 1 to 5 mols, preferably 1.2 to 4 mols, of cyanide group donor are added.

The inventive reaction takes place in an organic, aqueous or two-phase system or in emulsion in the presence of a hydroxynitrile lyase as catalyst.

In the enantioselective reaction in an aqueous system, an aqueous solution or buffer solution containing the corresponding HNL is used. Examples of these are acetate buffer, borate buffer, phthalate buffer, citrate buffer, phosphate buffer etc. or mixtures of these buffer solutions.

The pH of this solution is 1.5 to 5, preferably 2 to 4, particularly preferably 2.2 to 3.7.

In addition, water-miscible or immiscible solvents, for example, ethanol, DMF, toluene or t-butyl methyl ether can be added to the aqueous solution.

The organic diluent used can be water-immiscible or slightly water-miscible aliphatic or aromatic hydrocarbons which may be halogenated, alcohols, ethers or esters or mixtures thereof. Preferably, t-butyl methyl ether, diisopropyl ether, dibutyl ether and ethyl acetate or mixtures thereof are used.

However, the reaction can also proceed in a two-phase system or in emulsion.

Preferably, the inventive reaction takes place in an aqueous solution in the presence of a hydroxynitrile lyase as catalyst.

Suitable HNLs are not only native, but also recombinant (R)- and (S)-HNLs, which are present either as such or immobilized.

Suitable (S)-hydroxynitrile lyases are (S)-hydroxynitrile lyases, for example from manioc and Hevea brasiliensis, and recombinant (S)-HNLs. Preferably, the native HNL is that from Hevea brasiliensis. Suitable recombinant (S)-HNL is obtained, for example, from genetically modified microorganisms, for instance Pichia pastoris, E. coli or Saccharomyces cerevisiae.

Preference is given to recombinant (S)-HNL from Pichia pastoris.

Suitable (R)-HNLs are, for example, (R)-hydroxynitrile lyases from Prunus amygdalus, Prunus laurocerasus or Prunus serotina or recombinant (R)-HNLs. Preferably, the (R)-hydroxynitrilase from Prunus amygdalus, or a recombinant (R)-HNL is used.

Suitable (R)- and (S)-HNLs are disclosed, for example, by WO 97/03204, EP 0 969 095; EP 0 951 561, EP 0 927 766, EP 0 632 130, EP 0 547 655, EP 0 326 063, WO 01/44487 etc.

Preferably, recombinant (R)-HNL is used.

Per g of acetal or ketal, about 10 to 150 000 IU, preferably 1 200-40 000 IU, of activity of hydroxynitrile lyase are added.

The reaction temperature is about −5 to +40° C., preferably about 0 to +30° C.

The inventive process is suitable, in particular, for preparing those cyanohydrins whose corresponding carbonyl compounds are unstable. These are, for example, hydroxyaldehydes, which cyclize or polymerize spontaneously. The inventively prepared cyanohydrins are obtained here in high yields and with a higher enantiomeric excess compared with reactions using free aldehydes or ketones as substrate.

### EXAMPLE 1

5 ml of recombinant (R)-HNL solution (240 IU/ml) were adjusted to a pH of 2.4 using a citric acid solution. Then, 182 μl (1.1 mmol) of 1,1-dimethoxy-2-phenylethane and 0.1 ml (2.3 mmol) of prussic acid were added. The reaction solution was stirred at room temperature. The conversion rate and enantiomeric excess of the (R)-2-hydroxy-3-phenylpropanonitrile formed were determined by means of GC on a cyclodextrin column.

Course of Reaction:

(R)-2-Hydroxy-3-phenylpropanonitrile

| Hours | % conversion rate | % ee |
|---|---|---|
| 3 | 13 | 96.0 |
| 24 | 50 | 95.7 |
| 45.5 | 93 | 96.5 |

### EXAMPLE 2

185 μl (1.1 mmol) 3-chloro-1,1-diethoxypropane were added to 5 ml of recombinant (R)-HNL solution (430 IU/ml) having a pH of 2.4. 0.1 ml (2.3 mmol) of prussic acid was added to the mixture which was stirred at room temperature. After a reaction time of 20 hours the mixture was analyzed and (R)-3-chloro-2-hydroxybutanonitrile of >98% ee was found. The acetal had reacted completely.

### EXAMPLE 3

0.1 ml (2.3 mmol) of prussic acid was added at 0° C. to 5 ml of recombinant (R)-HNL solution (800 IU/ml) having a pH of 2.4. Then, 110 mg (0.55 mmol) of 4-hydroxy-β,β,5,5-tetramethyl-1,3-dioxane-2-ethanol (dimeric hydroxypivalaldehyde) were added and the mixture was stirred at 0° C. After a reaction time of 22 hours, 95% had reacted to form (R)-2,4-dihydroxy-3,3-dimethylbutanonitrile having 84% ee.

### EXAMPLE 4

### Comparison Example Using Aldehyde as Substrate

110 mg (0.55 mmol) of 4-hydroxy-β,β,5,5-tetramethyl-1,3-dioxane-2-ethanol (dimeric hydroxy-pivalaldehyde) were melted at 110° C. and monomerized for 40 minutes at this temperature. The 3-hydroxy-2,2-dimethylpropanal released was admixed with 5 ml of recombinant (R)-HNL solution (3 200 IU/ml) having a pH of 2.4. Then, the reaction was started by adding 0.1 ml (2.3 mmol) of prussic acid at 0° C. After a reaction time of 23.5 hours at 0° C., 97% had reacted to form (R)-2,4-dihydroxy-3,3-dimethylbutanonitrile having 70% ee.

1. A process for preparing enantiomer-enriched cyanohydrins using hydroxynitrile lyase (HNL) and a cyanide group donor which comprises reacting an acetal or ketal of the formula (I), 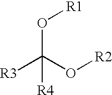
where
R1 and R2 independently of one another are an unsubstituted, monosubstituted or polysubstituted C₁-C₂₀-alkyl, C₅-C₂₀-aryl or C₅-C₂₀-heteroaryl radical,
or one of the two radicals is hydrogen, or R1 and R2 together form an unsubstituted, monosubstituted or polysubstituted C₂-C₂₀-alkylene radical,
and R3 and R4 independently of one another can be an unsubstituted, monosubstituted or polysubstituted C₁-C₂₀-alkyl, C₅-C₂₀-aryl, C₅-C₂₀-heteroaryl, C₇-C₂₀-alkylaryl, C₅-C₂₀-alkyl heteroaryl or C₅-C₂₀-aralkyl radical or an unsubstituted, monosubstituted or polysubstituted C₅-C₂₀-heterocycle or C₅-C₂₀-alkyl heterocycle or together can be an unsubstituted or substituted C₄-C₂₀-alkylene radical which can contain one or more heteroatoms in the chain, or one of the radicals is hydrogen,
in the presence of an (R)- or (S)-hydroxynitrile lyase and a cyanide group donor in an organic, aqueous or two-phase system or in emulsion at a temperature of −5 to +40° C. to give the corresponding enantiomer-enriched cyanohydrins of the formula (II), 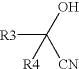
where
R3 and R4 are as defined above.

2. The process as claimed in claim 1, wherein acetals or ketals of the formula (I) are used as starting materials, where R1 and R2 independently of one another are a saturated, linear or branched C₁-C₁₂-alkyl radical which is unsubstituted, monosubstituted or polysubstituted by OH, halogen or phenyl, or one of the two radicals is hydrogen, or in which R1 and R2 together form a C₂-C₂₀-alkylene radical which can be unsubstituted, monosubstituted or polysubstituted by OH, halogen, phenyl or C₁-C₆-alkyl.

3. The process as claimed in claim 1, wherein acetals or ketals of the formula (I) are used as starting materials, where R3 and R4 independently of one another are a saturated or unsaturated, linear, branched or cyclic C₁-C₁₂-alkyl or phenyl radical which is unsubstituted, monosubstituted or polysubstituted by OH, halogen, phenyl, carboxylic esters, carboxamides, amino, Cₗ-C₆-alkylamino, C₆-C₂₀-arylamino, C₁-C₆-alkoxy, C₆-C₂₀-aryloxy or nitro, or one of the radicals is hydrogen, or where R3 and R4 together are a C₄-C₇-alkylene radical which is unsubstituted, monosubstituted or polysubstituted by OH, halogen, phenyl, carboxylic esters, carboxamides, amino, C₁-C₆-alkylamino, C₆-C₂₀-arylamino, C₁-C₆-alkoxy, C₁-C₆-alkyl, C₆-C₂₀-aryloxy or nitro, and which can contain one or two heteroatoms selected from the group consisting of O, N or S or an NR5R6 group, where R5 and R6 independently of one another can be H or C₁-C₆-alkyl.

4. The process as claimed in claim 1, wherein the enantioselective reaction is carried out in an aqueous system, where a solution or acetate buffer, borate buffer, phthalate buffer, citrate buffer or phosphate buffer solution containing the corresponding hydroxynitrile lyase, or mixtures of these buffer solutions, having a pH of 1.5 to 5 serves as reaction medium.

5. The process as claimed in claim 4, wherein a water-miscible or immiscible solvent selected from the group consisting of ethanol, t-butyl methyl ether, diisopropyl ether, dibutyl ether, dimethylformamide, toluene or ethyl acetate or mixtures thereof is added to the aqueous system.

6. The process as claimed in claim 1, wherein the hydroxynitrile lyase is a native or recombinant (R)- and (S)-hydroxynitrile lyase which is present either as such or immobilized.

7. The process as claimed in claim 6, wherein the hydroxynitrile lyase is a native (S)-hydroxynitrile lyase from manioc or Hevea brasiliensis, recombinant (S)-hydroxynitrile lyase from genetically modified microorganisms selected from the group consisting of Pichia pastoris, E. coli or Saccharomyces cerevisiae, native (R)-hydroxynitrile lyase from Prunus amygdalus, Prunus laurocerasus or Prunus serotina or recombinant (R)-hydroxynitrile lyase.

8. The process as claimed in claim 6, wherein a recombinant (R)-hydroxynitrile lyase is used.

9. The process as claimed in claim 1, wherein the cyanide group donor is prussic acid, alkali metal cyanides or cyanohydrins of the general formula (III),
R7R8C(OH)(CN)
where R7 and R8 independently of one another are hydrogen or an unsubstituted hydrocarbon group, or R7 and R8 together form an alkylene group having 4 or 5 carbon atoms, where R7 and R8 are not simultaneously hydrogen.

10. The process as claimed in claim 9, wherein the cyanide group donor is prussic acid, KCN, NaCN or acetone cyanohydrin.
